(19) United States
(12) Patent Application Publication
(10) Pub. No.: US 2021/0149222 A1
(43) Pub. Date: May 20, 2021

(54) Dental Shade-Matching Device

(71) Applicant: Jay Emirzian, Anderson, SC (US)

(72) Inventor: Jay Emirzian, Anderson, SC (US)

(21) Appl. No.: 17/099,712

(22) Filed: Nov. 16, 2020

Related U.S. Application Data
(60) Provisional application No. 62/935,557, filed on Nov. 14, 2019.

(51) Int. Cl. G02C 7/12; A61B 1/06; A61B 1/24

(52) U.S. Cl. CPC G02C 7/12; A61B 1/24; A61B 1/0692

(57) ABSTRACT

A dental shade-matching device is an apparatus that facilitates the viewing of teeth during medical examinations and/or procedures. The apparatus enhances the ability of a dentist or other medical practitioner to select an appropriate color shade of teeth, perform inspections and cleanings, and more. The apparatus can help dentists and other medical professionals to more clearly view teeth during practice without the visual obstruction of glare from the teeth. A potent light source provides light, preferably within an optimal range of luminous intensity. This light is filtered through a polarizing lens, which reduces the intensity of incident wavelengths of light upon the teeth of a patient. Light reflected from the teeth then passes through a set of polarized glasses, thus enabling further control over glare and incident light. This arrangement simultaneously illuminates target objects and filters out excess light and glare, thus providing an optimal view of desired objects.

The current application claims a priority to the U.S. Provisional Patent application Ser. No. 62/935,557 filed on Nov. 14, 2019. The current application is filed on Nov. 16, 2020 while Nov. 14, 2020 was on a weekend.

### FIELD OF THE INVENTION

The present invention relates generally to a dental tool. More specifically, the present invention relates to a wearable tool that directs polarized light toward an object, preferably a dental patient's teeth, and provides polarized lenses through which the operator may view the teeth. This arrangement enables reduction or elimination of glare from the patient's teeth.

### BACKGROUND OF THE INVENTION

Recreating nature in the dental field has proven to be a difficult engineering feat in a variety of categories and industries. Employing principles of biomimicry often results in the production of optimally designed items, such that desirable properties are promoted in products. For example, many geckos have the ability to stick to glass due to the microstructure of their palms and fingers; therefore, engineers have designed glass climbing tools based off of the design of gecko fingertips. Among the useful properties of light is its ability to become polarized under the appropriate conditions. Polarization reduces the intensity of incident wavelengths by restricting their wave motion to two dimensions, thus transforming light to be less damaging to the human eye without reducing the quality of the image that may be shown through the filter. Such polarization has also been found to have beneficial effects on images seen through them, including particularly the reduction of the intensity of reflected glare from items.

However, such developments have not been adequately explored and developed in many fields. Particularly in dental work, a medical professional may have to spend time viewing teeth under a bright light. Such light naturally causes unpleasant and even distracting reflections due to the translucency and opacity of teeth. Existing techniques for facilitating the selection of the color of teeth are often deficient, particularly as they relate to communicating dental tooth shades. There is a dearth of current technologies which can assist a user in viewing the internal and external color of teeth without glare or reflection. Many that attempt this feat do so using monitors and digitized images. This slows down communication during time-sensitive processes. Current technologies do not save valuable clinical time, nor do they save valuable time for technicians. What is needed is a tool that can help dentists and other medical professionals to more clearly view teeth during practice without the visual obstruction of glare from the teeth. Further desirable is a device that facilitates office operations and procedures.

The present invention addresses these issues. The dental shade-matching device facilitates viewing teeth during medical procedures. This enhances the ability of a practitioner to select an appropriate color shade of teeth, perform inspections and cleanings, and more. A potent light source provides light within an optimal range of luminous intensity. This light is filtered through a polarizing lens, which reduces the intensity of incident wavelengths of light upon the teeth of a patient. Light reflected from the teeth then passes through a set of polarized glasses, thus enabling further control over glare and incident light. This arrangement simultaneously illuminates target objects and filters out excess light and glare, thus eliminating internal and external reflections.

### BRIEF DESCRIPTION OF THE DRAWINGS

FIG. 1 is a perspective view of the present invention.

FIG. 2 is a top view of the present invention.

FIG. 3 is a front view of the present invention.

FIG. 4 is a front view of the present invention showing polarization lines.

FIG. 5 is a front view of the present invention showing polarization lines and multiple light sources.

FIG. 6 is a perspective view of the present invention showing a dimmer.

### DETAILED DESCRIPTION OF THE INVENTION

All illustrations of the drawings are for the purpose of describing selected versions of the present invention and are not intended to limit the scope of the present invention.

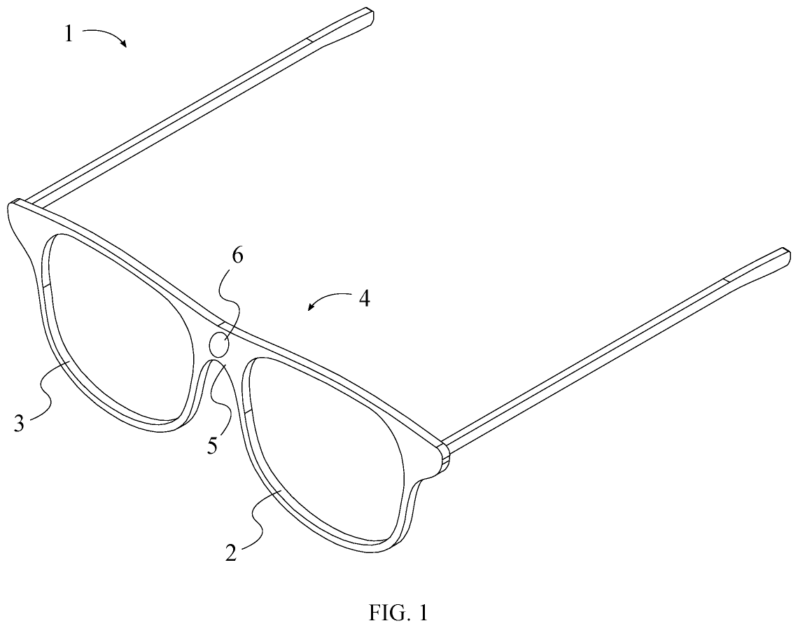

The present invention is a dental shade-matching device that is used to improve the ability of a dentist or other oral maintenance medical professional to view a patient's mouth during medical examinations and/or procedures. The present invention is configured to illuminate teeth by shining a light through at least one filter; the light that is subsequently reflected off the teeth is then filtered again to remove glare and improve overall image. The present invention comprises an eyewear 1 and at least one high-powered source of visible light 6, as shown in FIG. 1. The eyewear 1 relates to a set of glasses, goggles, spectacles, or other such ocular protective equipment capable of arranging lenses before a user's eye. The at least one high-powered source of visible light 6 is a controlled and directed light source, particularly an electrical bulb, capable of shining light in a desired direction. The eyewear 1 comprises a left optical-filtering lens 2, a right optical-filtering lens 3, and a frame 4. The left optical-filtering lens 2 is used to filter some portion of the light entering the user's left eye. Similarly, the right optical-filtering lens 3 is used to filter some portion of the light entering the user's right eye. The frame 4 is the rigid or semirigid support structure which secures the at least one high-powered source of visible light 6, the left optical-filtering lens 2, and the right optical-filtering lens 3 in appropriate position relative to each other, while simultaneously enabling the user to temporarily mount the present invention upon the user's face.

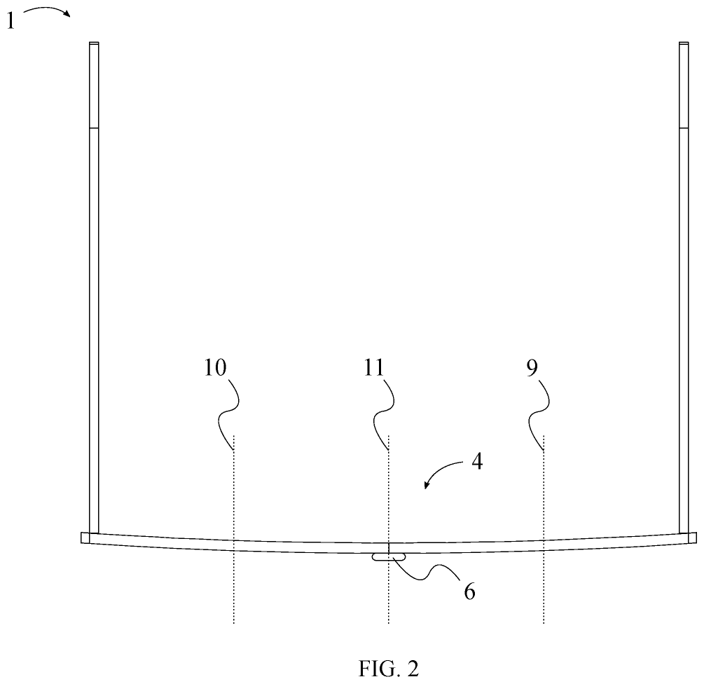

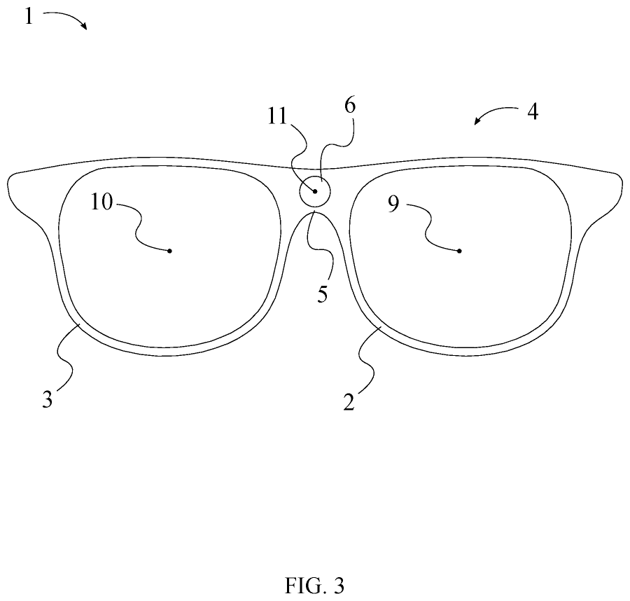

The general configuration of the aforementioned components allows the present invention to efficiently and effectively provide and subsequently adjust light to optimal viewable conditions. The left optical-filtering lens 2, the right optical-filtering lens 3, and the at least one high-powered source of visible light 6 are mounted onto the frame 4, as shown in FIGS. 2 and 3. This arrangement enables hands-free support of the left optical-filtering lens 2, the right optical-filtering lens 3, and the at least one high-powered source of visible light 6 by the user during use of the present invention. The left optical-filtering lens 2 and the right optical-filtering lens 3 are positioned offset from each other. In this way, the left optical-filtering lens 2 and the right optical-filtering lens 3 may each be dedicated to the respective eye of the user. A left viewing axis 9 of the left optical-filtering lens 2 and a right viewing axis 10 of the right optical-filtering lens 3 are positioned parallel to each other. This arrangement ensures optimal conditions for light manipulation through the left optical-filtering lens 2 and the right optical-filtering lens 3. An emitting axis 11 of the at least one high-powered source of visible light 6 is positioned parallel to the left viewing axis 9 and the right viewing axis 10. Thus, light emitted from the at least one high-powered source of visible light 6 is oriented optimally for subsequent reflection into the left optical-filtering lens 2 and the right optical-filtering lens 3.

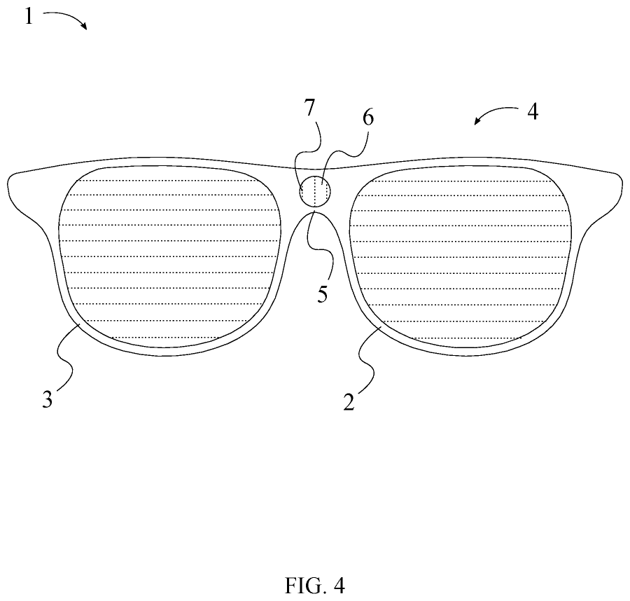

The present invention requires a mechanism by which to affect the outputted light from the at least one high-powered light source. To this end, the present invention may further comprise at least one optical filter 7, as shown in FIG. 4. The at least one optical filter 7 relates to a series of thin, transparent interfaces through which light can be modified, including any or any combination of polarizing lenses, tinted lenses, spectral filters, and more. In an exemplary embodiment, the at least one optical filter 7 is a polarizing lens. The at least one optical filter 7 is mounted onto the at least one high-powered source of visible light 6. In this way, the at least one optical filter 7 is positioned at a constant distance and orientation from the at least one high-powered source of visible light 6. The emitting axis 11 traverses through the at least one optical filter 7. This arrangement ensures that light emitted from the at least one high-powered source of visible light 6 is oriented to travel through the at least one optical filter 7. A left-lens polarization of the left optical-filtering lens 2 and a right-lens polarization the right optical-filtering lens 3 are oriented parallel to each other. Thus, light passing through the left optical-filtering lens 2 is functionally identical to light passing through the right optical-filtering lens 3. A filter polarization of the at least one optical filter 7 is oriented perpendicular to the left-lens polarization. This arrangement results in reflected light being reduced to minimal intensity and noise when passing through the left optical-filtering lens 2. Similarly, the filter polarization is oriented perpendicular to the right-lens polarization. This arrangement results in reflected light being reduced to minimal intensity and noise when passing through the right optical-filtering lens 3.

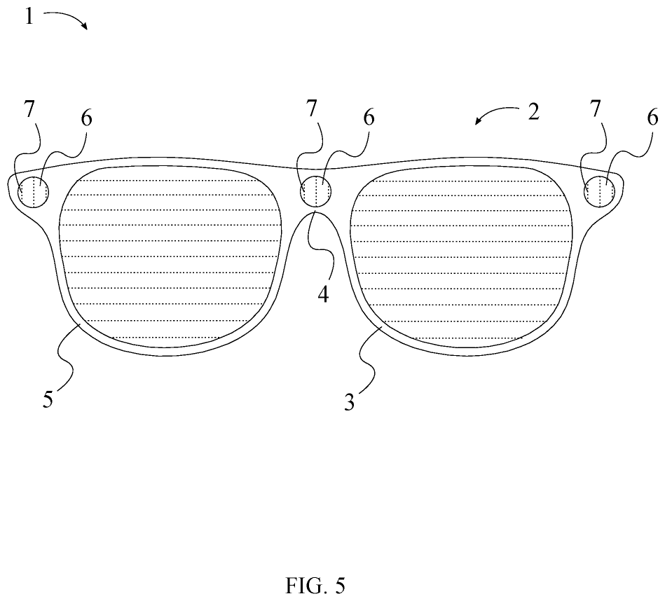

It is possible that a single source of light is not ideal for providing an optimal, shadow-minimizing image of a mouth. To mitigate this possibility, the at least one optical filter 7 may be a plurality of optical filters, as shown in FIG. 5. This allows the at least one optical filter 7 to cover a variety of different light sources. Furthermore, the at least one high-powered source of visible light 6 may be a plurality of high-powered sources, wherein each of the plurality of high-powered sources is associated with a corresponding optical filter from the plurality of optical filters. In this way, the present invention may provide illumination from a variety of directions, thus reducing or eliminating the presence of shadows in an image or view. The emitting axis 11 of each of the plurality of high-powered sources traverses through the corresponding optical filter. This arrangement ensures that emitted light from each of the plurality of high-powered sources will also traverse through the corresponding optical filter. The filter polarization of the corresponding optical filter for each of the plurality of high-powered sources is oriented parallel to each other. Thus, emitted light is polarized in the same direction from each of the plurality of high-powered sources, ensuring uniform properties of light incident upon the patient's teeth.

Optimal viewing light for illuminating images and objects exists within a range on the electromagnetic spectrum. To provide a user with optimal lighting, a color temperature of the at least one high-powered source of visible light 6 may be approximately in between 5000 kelvins (K) and 6000 K. This range has been identified as being most similar to daylight and thus represents a preferable range for the luminous intensity of lighting sources for humans. The light can vary in intensity depending on the preference of the dental professional.

The eyewear 1 must have a mechanism for connecting to a user's face while simultaneously arranging the left optical-filtering lens 2 and the right optical-filtering lens 3. To this end, the eyewear 1 may further comprise a nose bridge 5, as shown in FIG. 1. The nose bridge 5 is a connective rigid segment capable of conforming to the shape of a nose, thus leveraging the nose as a balance and support mechanism for the eyewear 1. The left optical-filtering lens 2 is positioned adjacent to the nose bridge 5. This arrangement allows the nose bridge 5 to support the left optical-filtering lens 2. Similarly, the right optical-filtering lens 3 is positioned adjacent to the nose bridge 5, opposite the left optical-filtering lens 2. Thus, the left optical-filtering lens 2 and the right optical-filtering lens 3 are positioned apart from each other and connected by the nose bridge 5. The at least one high-powered source of visible light 6 is positioned adjacent to the nose bridge 5 in between the left optical-filtering lens 2 and the right optical-filtering lens 3. In this way, the at least one high-powered source of visible light 6 provides even and equal lighting to both the left optical-filtering lens 2 and the right optical-filtering lens 3 during a preferred usage of the present invention.

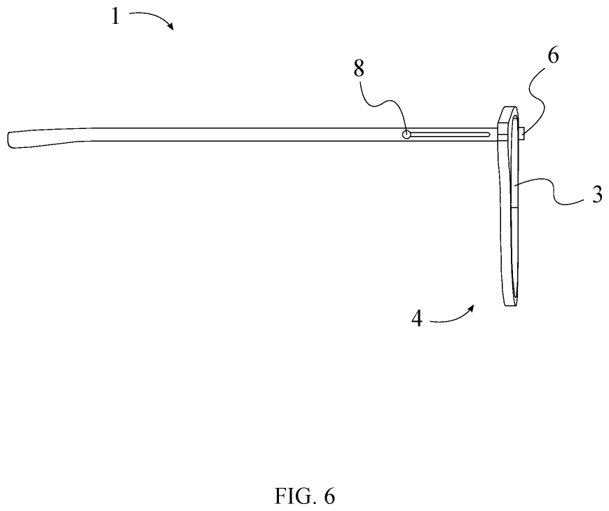

The at least one high-powered source of visible light 6 may require adjustment relative to time of day, ambient lighting, and other environmental conditions or user preferences. To enable this, the present invention may further comprise a dimmer 8, as shown in FIG. 6. The dimmer 8 is a slider, knob, touchscreen interface, or other such control system capable of enabling the user to interact with the brightness setting of the at least one high-powered source of visible light 6. The dimmer 8 is operatively coupled to the at least one high-powered source of visible light 6, wherein the dimmer 8 is used to adjust an illumination intensity of the at least one high-powered source of visible light 6. This arrangement ensures that the user may have control over the intensity of light incident upon an object, especially the mouth of a patient.

The user may wish to examine objects more closely while using the present invention. To this end, the left optical-filtering lens 2 and the right optical-filtering lens 3 may be a pair of magnifying lenses with a specified diopter. This arrangement ensures that the user may give special attention to details and specific features of the object or objects being observed.

Although the invention has been explained in relation to its preferred embodiment, it is to be understood that many other possible modifications and variations can be made without departing from the spirit and scope of the invention as hereinafter claimed.

What is claimed is:

1. A dental shade-matching device comprises:
an eyewear;
at least one high-powered source of visible light;
the eyewear comprises a left optical-filtering lens, a right optical-filtering lens, and a frame;
the left optical-filtering lens, the right optical-filtering lens, and the at least one high-powered source of visible light being mounted onto the frame;
the left optical-filtering lens and the right optical-filtering lens being positioned offset from each other;
a left viewing axis of the left optical-filtering lens and a right viewing axis of the right optical-filtering lens being positioned parallel to each other; and
an emitting axis of the at least one high-powered source of visible light being positioned parallel to the left viewing axis and the right viewing axis.

2. The dental shade-matching device as claimed in claim 1 comprises:
at least one optical filter;
the at least one optical filter being mounted onto the at least one high-powered source of visible light;
the emitting axis traversing through the at least one optical filter;
a left-lens polarization of the left optical-filtering lens and a right-lens polarization the right optical-filtering lens being oriented parallel to each other;
a filter polarization of the at least one optical filter being oriented perpendicular to the left-lens polarization; and
the filter polarization being oriented perpendicular to the right-lens polarization.

3. The dental shade-matching device as claimed in claim 2 comprises:
the at least one optical filter being a plurality of optical filters;
the at least one high-powered source of visible light being a plurality of high-powered sources, wherein each of the plurality of high-powered sources is associated with a corresponding optical filter from the plurality of optical filters;
the emission axis of each of the plurality of high-power sources traversing through the corresponding optical filter; and
the filter polarization of the corresponding optical filter for each of the plurality of high-powered sources being oriented parallel to each other.

4. The dental shade-matching device as claimed in claim 1, wherein a color temperature of the at least one high-powered source of visible light is approximately in between 5000 kelvins (K) and 6000 K.

5. The dental shade-matching device as claimed in claim 1 comprises:
the eyewear further comprises a nose bridge;
the left optical-filtering lens being positioned adjacent to the nose bridge;
the right optical-filtering lens being positioned adjacent to the nose bridge, opposite the left optical-filtering lens; and
the at least one high-powered source of visible light being positioned adjacent to the nose bridge in between the left optical-filtering lens and the right optical-filtering lens.

6. The dental shade-matching device as claimed in claim 1 comprises:
a dimmer; and
the dimmer being operatively coupled to the at least one high-powered source of visible light, wherein the dimmer is used to adjust an illumination intensity of the at least one high-powered source of visible light.

7. The dental shade-matching device as claimed in claim 1, wherein the left optical-filtering lens and the right optical-filtering lens are a pair of magnifying lenses with a specified diopter.

8. A dental shade-matching device comprises:
an eyewear;
at least one high-powered source of visible light;
at least one optical filter;
the eyewear comprises a left optical-filtering lens, a right optical-filtering lens, and a frame;
the left optical-filtering lens, the right optical-filtering lens, and the at least one high-powered source of visible light being mounted onto the frame;
the left optical-filtering lens and the right optical-filtering lens being positioned offset from each other;
a left viewing axis of the left optical-filtering lens and a right viewing axis of the right optical-filtering lens being positioned parallel to each other;
an emitting axis of the at least one high-powered source of visible light being positioned parallel to the left viewing axis and the right viewing axis;
the at least one optical filter being mounted onto the at least one high-powered source of visible light;
the emitting axis traversing through the at least one optical filter;
a left-lens polarization of the left optical-filtering lens and a right-lens polarization the right optical-filtering lens being oriented parallel to each other;
a filter polarization of the at least one optical filter being oriented perpendicular to the left-lens polarization; and
the filter polarization being oriented perpendicular to the right-lens polarization.

9. The dental shade-matching device as claimed in claim 8 comprises:
the at least one optical filter being a plurality of optical filters;
the at least one high-powered source of visible light being a plurality of high-powered sources, wherein each of the plurality of high-powered sources is associated with a corresponding optical filter from the plurality of optical filters;
the emission axis of each of the plurality of high-power sources traversing through the corresponding optical filter; and
the filter polarization of the corresponding optical filter for each of the plurality of high-powered sources being oriented parallel to each other.

10. The dental shade-matching device as claimed in claim 8, wherein a color temperature of the at least one high-powered source of visible light is approximately in between 5000 K and 6000 K.

11. The dental shade-matching device as claimed in claim 8 comprises:
the eyewear further comprises a nose bridge;
the left optical-filtering lens being positioned adjacent to the nose bridge;
the right optical-filtering lens being positioned adjacent to the nose bridge, opposite the left optical-filtering lens; and
the at least one high-powered source of visible light being positioned adjacent to the nose bridge in between the left optical-filtering lens and the right optical-filtering lens.

12. The dental shade-matching device as claimed in claim 8 comprises:
a dimmer; and
the dimmer being operatively coupled to the at least one high-powered source of visible light, wherein the dimmer is used to adjust an illumination intensity of the at least one high-powered source of visible light.

13. The dental shade-matching device as claimed in claim 8, wherein the left optical-filtering lens and the right optical-filtering lens are a pair of magnifying lenses with a specified diopter.

14. A dental shade-matching device comprises:
an eyewear;
at least one high-powered source of visible light;
the eyewear comprises a left optical-filtering lens, a right optical-filtering lens, a frame, and a nose bridge;
the left optical-filtering lens, the right optical-filtering lens, and the at least one high-powered source of visible light being mounted onto the frame;
the left optical-filtering lens and the right optical-filtering lens being positioned offset from each other;
a left viewing axis of the left optical-filtering lens and a right viewing axis of the right optical-filtering lens being positioned parallel to each other;
an emitting axis of the at least one high-powered source of visible light being positioned parallel to the left viewing axis and the right viewing axis;
the left optical-filtering lens being positioned adjacent to the nose bridge;
the right optical-filtering lens being positioned adjacent to the nose bridge, opposite the left optical-filtering lens; and
the at least one high-powered source of visible light being positioned adjacent to the nose bridge in between the left optical-filtering lens and the right optical-filtering lens.

15. The dental shade-matching device as claimed in claim 14 comprises:
at least one optical filter;
the at least one optical filter being mounted onto the at least one high-powered source of visible light;
the emitting axis traversing through the at least one optical filter;
a left-lens polarization of the left optical-filtering lens and a right-lens polarization the right optical-filtering lens being oriented parallel to each other;
a filter polarization of the at least one optical filter being oriented perpendicular to the left-lens polarization; and
the filter polarization being oriented perpendicular to the right-lens polarization.

16. The dental shade-matching device as claimed in claim 15 comprises:
the at least one optical filter being a plurality of optical filters;
the at least one high-powered source of visible light being a plurality of high-powered sources, wherein each of the plurality of high-powered sources is associated with a corresponding optical filter from the plurality of optical filters;
the emission axis of each of the plurality of high-power sources traversing through the corresponding optical filter; and
the filter polarization of the corresponding optical filter for each of the plurality of high-powered sources being oriented parallel to each other.

17. The dental shade-matching device as claimed in claim 14, wherein a color temperature of the at least one high-powered source of visible light is approximately in between 5000 K and 6000 K.

18. The dental shade-matching device as claimed in claim 14 comprises:
a dimmer; and
the dimmer being operatively coupled to the at least one high-powered source of visible light, wherein the dimmer is used to adjust an illumination intensity of the at least one high-powered source of visible light.

19. The dental shade-matching device as claimed in claim 14, wherein the left optical-filtering lens and the right optical-filtering lens are a pair of magnifying lenses with a specified diopter.
